(19) United States
(12) Patent Application Publication
(10) Pub. No.: US 2013/0071480 A1
(43) Pub. Date: Mar. 21, 2013

(54) Particles which are surface coated with hyaluronan or one of the derivatives thereof and the use of same as biological vectors for active substances

(71) Applicant: Centre National De La Recherche Scientifique, Paris (FR)

(72) Inventors: Edith DELLACHERIE, Malzeville (FR); Michele Leonard, Chaligny (FR); Ruxandra Gref, Le Plessis Robinson (FR); Patrick Netter, Nancy (FR); Elisabeth Payan, Trondes (FR)

(73) Assignee: Centre National De La Recherche Scientifique, Paris (FR)

(21) Appl. No.: 13/675,734

(22) Filed: Nov. 13, 2012

Related U.S. Application Data
(63) Continuation of application No. 13/137,283, filed on Aug. 3, 2011, now abandoned.
Application No. 13/137,283 is a continuation of application No. 10/522,333, filed on May 24, 2005, now abandoned.

(30) Foreign Application Priority Data
Jul. 25, 2002 (FR) 02-09436

(51) Int. Cl. A61K 9/50

(52) U.S. Cl. CPC A61K 9/5036; A61K 9/5089
USPC 424/493; 514/1.1; 514/23; 514/44 R; 514/54; 424/184.1; 424/94.1; 264/4.6

(57) ABSTRACT

Particles having a core based on at least one biodegradable organosoluble polymer. At least a part of the surface of the particles is coated with at least one hyaluronan or a derivative thereof, the hyaluronan being a water-soluble, amphiphilic hyaluronan of which the carboxylic functions are in part transformed to form hydrophobic groups.

This is a continuation of application Ser. No. 13/137,283 filed Aug. 3, 2011, which is a continuation of application Ser. No. 10/522,333 filed May 24, 2005, which is a National Stage Application of PCT/FR03/02299 filed Jul. 21, 2003, and claims the benefit of French Application No. 02 09436 filed Jul. 25, 2002. The entire disclosures of the prior applications are hereby incorporated by reference herein in their entirety.

The present invention relates mainly to particles which are at least partially surface-coated with hyaluronan or a derivative, and the use of these particles as biological vectors for active substances,

Vectorization is an operation aimed at modulating and if possible completely controlling the distribution of a substance by associating it with a suitable system called a vector.

In the vectorization field, three main functions must be performed:
transporting the active substance(s) in the biological fluids of the organism,
conveying the active substances to the organs to be treated, and
ensuring the release of these active substances.

Added to these three functions is a vector bioavailability requirement. The vector must be biodegradable and its subunits must be tolerated by the organism.

In fact, the outcome of the vector, in vivo, is conditioned by its size, its physicochemical characteristics and, in particular, its surface properties which, firstly, play a determining role with the components of the biological medium and, secondly, can induce behavior targeted to a specific site to be treated.

The biological vectors more particularly concerned in the context of the present invention belong to the field of particles, in particular nanoparticles and microparticles. Nanoparticles and microparticles of poly(lactic acid) (PLA) and/or of biodegradable polyester, the degradation products of which are natural metabolites of a human organism, have for a long time been proposed for vectorizing bioactive molecules for various types of administration. However, the hydrophobic nature of the surface of these particles and the presence of carboxylate groups (ends of the PLA chains) result in an adsorption of plasma proteins, opsonins, responsible in particular for uptake of the particles by cells of the Mononuclear Phagocyte System (MPS), As a result of this, the particles disappear rapidly from the circulating volume and at the same time accumulate in the organisms of the MPS (liver, spleen, kidneys).

The objective of the present invention is in particular to propose novel particles that have a prolonged lifetime and are particularly advantageous for carrying biological or synthetic active substances, that are advantageous in the rheumatology field.

In this clinical field, the practitioner is often confronted with inflammatory and/or degenerative pathologies which engender, in the more or less long term, cartilage degradation that is sometimes irreversible. Besides non-specific treatments based on analgesics and nonsteroidal anti-inflammatories, use may be made, inter alia, of local injections of corticosteroids, The high doses of corticosteroids used in this situation can, however, induce not insignificant adverse effects. Moreover, it is generally necessary to multiply these injections due to beneficial action not being great enough.

The administration of this type of active substances by means of particles would therefore be a particularly advantageous alternative to conventional therapies.

In this case, the present invention aims in particular to propose a particle-type vector which, firstly, is biodegradable and suitable for the controlled release of an active substance and, secondly, is capable of effectively targeting the release of this active substance at tissue cells, and more particularly cells having hyaluronan-specific receptors, also called CD44.

These receptors are in particular present on cells of the joint region, such as for example chondrocytes and synoviocytes. Chondrocytes are cells involved in the synthesis of the cartilaginous matrix, They also control the maintenance of cartilage homeostasis. Synoviocytes, which are cells located in the synovial membrane, are for their part involved in the synthesis of hyaluronan in the synovial fluid,

Unexpectedly, the inventors have demonstrated that it is possible to effectively target the release of an active substance encapsulated in particles to cells having in particular this type of receptor, by surface-functionalizing them with hyaluronan.

A first aspect of the invention therefore concerns particles in which the core is based on at least one biodegradable organosoluble polymer, characterized in that they are at least partially surface-coated with at least one hyaluronan or with one of its derivatives.

Hyaluronic acid is a natural polysaccharide consisting of a series of N-acetylglucosamine/glucuronic acid disaccharide units, aqueous solutions of which have a high viscosity, It is present in particular in the umbilical cord, in the vitreous humor and in the synovial fluid. It is also produced by certain bacteria, in particular by hemolytic streptococci of groups A and C. The molar mass of hyaluronic acid can range from 10 000 to 10 000 000 g approximately, according to the origin. Hyaluronic acid is in particular sold in the form of its sodium salt (also called hyaluronate). The generic term “hyaluronan” is used to denote, without distinction, hyaluronic acid and hyaluronates, especially in the form of inorganic or organic salts, and in particular alkali metal salts and/or alkaline-earth metal salts.

More particularly, this hyaluronan is used in the form of a water-soluble amphiphilic hyaluronan, the carboxylic functions of which are in part converted so as to form hydrophobic groups. The attachment of these hydrophobic groups can in particular be established by reaction thereof with the carboxylic functions of the hyaluronate according to an esterification or amidation reaction. This conversion is carried out to a degree sufficient to confer amphiphilic behavior on said hyaluronan.

The conversion of the carboxylic functions of the hyaluronate can thus be obtained by partial esterification and/or amidation of these functions. Such derivatives are in particular described in FR 2 791763.

The hydrophobic groups can in particular derive from esterification of the carboxylic functions with at least one group selected from the group consisting of:
linear or branched, saturated or unsaturated alkyl chains which may be interrupted with one or more hetero atoms such as S, O and N atoms and, where appropriate, substituted with at least one aromatic ring, and
oligomers such as those that derive from α-hydroxy acids.

The alkyl chains may have a number of carbon atoms of greater than 5, and in particular greater than 10. However, in the specific case where such a chain is substituted with an aromatic ring, its number of carbon atoms may be smaller. The degree of conversion is generally adjusted so as to preserve sufficient water-solubility for the amphiphilic hyaluronan derivative thus obtained.

It is also controlled by taking into account the hydrophobicity of the groups attached to the hyaluronan.

In general, as regards the alkyl chains, the longer the chain, the lower the degree of attachment on the hyaluronan backbone may be. Conversely, with short alkyl chains, the degree of attachment may be higher. It is clear that this adjustment between the degree of attachment and the length of the alkyl chains is within the competence of those skilled in the art.

By way of indication, for an alkyl chain containing from 15 to 20 carbon atoms, and in particular 18 carbon atoms, the degree of esterification may be at most 15%, or even less than 10%, and especially less than 7%, and in particular between 0.05 and 5%. For an alkyl chain containing from 10 to 14 carbon atoms, and in particular 12 carbon atoms, this degree of esterification may be greater than or equal to 25%, and for an alkyl chain of 6 carbon atoms, it may be of the order of 50%.

This degree of conversion is generally adjusted so as to allow attachment of the hyaluronan derivative at the surface of the particles by means of the interaction of its hydrophobic groups with the hydrophobic polymeric matrix constituting the particles. In other words, the alkyl chains are anchored in the hydrophobic matrix during the formation of the particles. In the case of the present invention, the surface-functionalization of the particles with hyaluronan does not involve either a covalent or an ionic bond between these two entities. The attachment of the hyaluronan derivative is essentially the result of interactions of the hydrophobic and Van der Waals type. The degree of conversion is also adjusted so as not to affect the natural affinity of the hyaluronan for CD44 receptors.

The presence of hyaluronan at the surface of the particles is particularly advantageous for selectively directing them to the CD44 receptors present in particular on the cells of the joint region. By virtue of this hyaluronan envelope, the particles according to the invention, used as a biological vector for a biological or synthetic active substance, advantageously make it possible to effectively target the release of this active substance at cells possessing CD44 receptors, for example chondrocytes and/or synoviocytes, This results in a controlled and prolonged action of this active substance at the targeted lesion. The latter aspect is particularly advantageous for the patient's well-being, in so far as it provides access to better availability of the medicinal product and therefore makes it possible to reduce the amounts administered and the frequency of administration thereof.

The biodegradability of the claimed particles is, moreover, also provided by virtue of the nature of the polymers of which they are formed.

For the purpose of the invention, the term “biodegradable” is intended to denote any polymer which dissolves or degrades over a period of time acceptable for the application for which it is intended, usually in in vivo therapy. Generally, this period of time should be less than 5 years, and more preferably less than a year, when a corresponding physiological solution is exposed to a pH of 6 to 8 and to a temperature of between 25° C. and 37° C.

The biodegradable polymers according to the invention are, or are derived from, synthetic or natural biodegradable polymers.

As regards the organosoluble biodegradable polymers that can be used to constitute the core of the particles, they may in particular be selected from the group consisting of polyesters such as poly(lactic acid) (PLA), poly(glycolic acid) (PGA) or poly(ε-caprolactone) (PCL), polyanhydrides, poly(alkyl cyanoacrylates), polyorthoesters, poly(alkylene tartrate), polyphosphazenes, polyamine acids, polyamidoamines, polycarbonates, poly(methylidene malonate), polysiloxane, polyhydroxybutyrate or poly(malic acid), and also their copolymers and derivatives.

According to a particular variant of the invention, the claimed particles have a polymeric matrix incorporating at least one polymer different from hyaluronan. More preferably, this matrix consists of one or more polymers other than hyaluronan.

Particularly preferred as biodegradable organosoluble polymers according to the invention are polyesters such as polylactic acid), poly(glycolic acid) or polys-caprolactone), and their copolymers, such as, for example, poly(lactic acid-co-glycolic acid) (PLGA).

According to a particular variant of the invention, the particles are composed mainly, i.e. at more than 50% by weight, in particular more than 75% by weight, or even entirely, of polylactic acid)

The particles according to the invention preferably comprise at least one biological or synthetic active substance of the medicinal product type, in a form encapsulated in the polymer core.

As biological active substances, mention may more particularly be made of peptides, proteins, carbohydrates, nucleic acids, lipids, polysaccharides or mixtures thereof. They may also be synthetic organic or inorganic molecules which, when administered in vivo to an animal or to a patient, are capable of inducing a biological effect and/or manifesting a therapeutic activity. They may thus be antigens, enzymes, hormones, receptors, vitamins and/or minerals.

As a nonlimiting representation of the medicinal products that may be incorporated into these particles, mention may be made of anti-inflammatory compounds, anesthetics, chemotherapeutic agents, immunotoxins, immunosuppressants, steroids, antibiotics, antiviral agents, antifungal agents, antiparasitic agents, immunizing substances, immunomodulators and analgesics.

In so far as the particles according to the invention are advantageously targeted to tissue structural cells such as, for example, chondrocytes and synoviocytes, the use of the following compounds as active substances may be favored: anti-inflammatories, matrix components such as, for example, glycosaminoglycans and biological factors involved in the process of regeneration and/or protection of cartilage. The particles have in particular the advantage of effectively protecting this type of active substance particularly sensitive to the biodegradation phenomenon.

They may also be biological compounds that are more particularly active with respect to arthrosis, such as, for example, glucosamine from glycosaminoglycans, hyaluronic acid, chondroitin sulfate and mixtures thereof.

The particles in accordance with the invention may comprise up to 95% by weight of an active substance.

The active substance may thus be present in an amount ranging from 0.001 to 950 mg/g of particle, and preferably from 0.1 to 500 mg/g. It should be noted that, in the case of the encapsulation of certain macromolecular compounds (DNA, oligonucleotides, proteins, peptides, etc.), even lower loads may be sufficient.

The particles according to the invention may have a size ranging from 50 nm to 600 μm, and in particular from 80 nm to 250 μm.

The particles according to the invention having a size of between 1 and 1000 nm are called nanoparticles. The particles for which the size ranges from 1 to several thousand microns are referred to as microparticles.

The particles are generally of spherical shape, but may also be in other shapes.

The claimed nanoparticles or microparticles can be prepared according to methods already described in the literature, and can more particularly be obtained by means of the emulsion/solvent evaporation technique, and especially that described by R. Gurny et al. “Development of biodegradable and injectable latices for controlled release of potent drugs” Drug Dev, Ind. Pharm., vol, 7, p. 1-25 1981.

Unexpectedly, the inventors have thus demonstrated that the abovementioned amphiphilic hyaluronans can advantageously be used instead of the conventional surfactants for preparing particles according to the emulsion/solvent evaporation technique.

In fact, two variants of this technique are considered according to the hydrophobic or hydrophilic nature of the active substance to be encapsulated.

When it is sought to encapsulate a hydrophobic active substance, a single emulsion is prepared. To do this, the selected biodegradable polymer is dissolved in the organic phase, in particular a solvent that is barely soluble in water, such as for example methylene chloride or ethyl acetate, with the active substance to be encapsulated. The amphiphilic hyaluronan is, for its part, dissolved in the aqueous phase which serves as a dispersing medium for the organic phase. After mixing of these two phases, the hyaluronan derivative is located at the water/organic phase interface due to its amphiphilic properties, and thus stabilizes the emulsion. When the organic solvent is evaporated off, the amphiphilic hyaluronan derivatives remain advantageously attached at the surface of the particles thus formed, the hydrophobic groups being anchored more or less deeply in the organosoluble polymer core farming the particles, and the hydrophilic component, mainly corresponding to the hyaluronan backbone, being exposed at the surface. Once solvent evaporation is complete, particles in accordance with the invention are recovered and are subsequently subjected to washing with water, centrifugation or lyophilization.

When the active substance to be encapsulated is hydrophilic, like proteins and polysaccharides for example, a first emulsion of the oil-in-water type is prepared, composed of an organic phase containing the biodegradable organosoluble polymer and of a first aqueous phase containing the active substance. This “inverse” emulsion is then brought together with a second aqueous phase containing the amphiphilic hyaluronan derivative, so as to obtain a water/oil/water double emulsion with respect to which the amphiphilic hyaluronan acts as a stabilizer, After solvent evaporation, particles in accordance with the present invention are recovered and are treated as above.

The conditions used during the preparation of the emulsions generally determine the size of the particles, and the adjustment of these conditions is within the competence of those skilled in the art.

The use of a hyaluronan derivative as a stabilizing agent in this method of preparing particles is therefore particularly advantageous in at least two respects:
it makes it possible to do without the presence of the surfactants systematically used in conventional methods. In this case, the latter are not always biocompatible and are sometimes difficult to remove at the end of synthesis;
it results, at the end of the synthesis of the particles, in a vector that exhibits selective affinity for tissue structural cells, and more particularly for cells of the joint region, and in particular for chondrocytes and synoviocytes.

The concentration of hyaluronan in the medium for synthesizing the particles determines the degree of coating of the particles, i.e. the amount of hyaluronan deposited at their surface. The hyaluronan derivative is generally evenly distributed over the surface of the particles, with a surface density that can vary significantly.

It is also possible to incorporate compounds for diagnostic purposes into the particles. They may thus be substances that can be detected by X-rays, fluorescence, ultrasound, nuclear magnetic resonance or radioactivity. The particles may thus include magnetic particles, radio-opaque materials, such as in particular barium, or fluorescent compounds. Alternatively, gamma-emitters (for example indium or technetium) may be incorporated therein.

As described above, the active substance is preferably incorporated into these particles during their formation process. However, when it proves to be possible, they may also be loaded into the particles once said particles have been obtained.

The particles according to the invention can be administered in various ways, for example orally or parenterally, and in particular via the intra-articular, ocular, pulmonary, nasal, vaginal, cutaneous and/or buccal routes.

Since the hyaluronans present at the surface of the particles according to the invention bear a multitude of reactive OH functions, it is also possible, once the particles have been formed, to attach all sorts of molecules to these functions, via covalent bonds. By way of nonlimiting illustration of this type of molecules, mention may in particular be made of molecules of label type and compounds capable of potentiating the targeting function performed by the hyaluronan, such as, for example, RGD (arginine-glycine-aspartic acid) peptides which promote adhesion between cells and their extracellular matrices.

A second aspect of the invention concerns a biological vector, in particular for one or more biological or synthetic active substance(s), comprising at least particles according to the invention.

The invention also relates to the use of this vector, or of the claimed particles, for encapsulating at least one biological or synthetic active substance.

Another aspect of the invention relates to pharmaceutical or diagnostic compositions comprising at least one vector and in particular particles according to the invention, where appropriate combined with at least one pharmaceutically acceptable and compatible carrier.

As mentioned above, the claimed particles are particularly advantageous in pharmaceutical or diagnostic terms.

They provide satisfactory protection of the encapsulated active substance. They limit the diffusion of this active substance in the organism by virtue of the steric effect of the particles per se, and of the natural affinity of the hyaluronan for CD44 receptors. They allow gradual release of this active substance in the vicinity of the lesion and/or of the cells targeted, thus permitting a prolonged action. Finally, they degrade slowly into products that are well tolerated by the organism.

Another aspect of the present invention concerns the use of particles as defined above or even of a biological vector incorporating them, for preparing a pharmaceutical composition intended for the treatment of arthrosis.

The particles can also be incorporated into capsules, or incorporated into implants, gels or lozenges. They can also be formulated directly in a fluid of the oil type, for example, and can be injected directly into the biological site to be treated.

Another aspect of the invention concerns the use of amphiphilic hyaluronan or derivative as defined above, as a targeting agent at the surface of particles consisting in particular of at least one biodegradable polymer, or of capsules, in particular hollow capsules. These particles or capsules may in particular be nano spheres or microspheres, or nanoparticles or microparticles.

The examples and figures presented hereinafter are given by way of nonlimiting illustration of the field of the invention.

### FIGURES

FIG. 1: Representation of the cell proliferation of rat chondrocytes cultured in a monolayer system, after treatment for 48 h in the presence of nanoparticles based on PLA (control), and on PLA coated with amphiphilic hyaluronan.

FIG. 2: Representation of the cell proliferation and proteoglycan synthesis activity of chondrocytes cultured on alginate beads (three-dimensional culture), after treatment for 48 h in the presence of nanoparticles based on PLA (control), and on PLA coated with amphiphilic hyaluronan.

### MATERIALS AND METHODS

Synthesis of modified HAs

By way of example, the synthesis of a hyaluronan substituted with aliphatic chains containing 18 carbons is described below.

The sodium hyaluronate (HA, M_{w}=600 000 g/mol) comes from the company Bioibérica (Barcelona, Spain).

1 g of sodium hyaluronate is dissolved in 100 ml of distilled water. The solution is brought into contact, for 15 minutes, with 5 g of Dowex 50*8 cation exchange resin conditioned with H⁺ at 2.5 meq/g (stoichiometry 1:6). After filtration, the solution containing the acid form of the polysaccharide is neutralized at pH 7 with tetrabutylammonium hydroxide, and then lyophilized. Tetrabutylammonium hyaluronate, HA-TBA, is thus obtained.

1 g of HA-TBA is dissolved in 100 ml of dimethyl sulfoxide. 36 μl of C₁₈H₃₇Br are added. After reaction for 24 h at 30° C. with stirring, the mixture is dialyzed: 1 day against distilled water, 6 days against distilled water+azide NaN₃ ( 1/2500), and then 1 day against distilled water. Finally, the dialyzed solution is lyophilized.

A sodium hyaluronate derivative in which approximately 4% of the carboxylic functions are esterified with chains containing 18 carbons is thus obtained.

### Example 1

### Synthesis and Characterization of Particles in Accordance with the Invention

By way of example, the protocol for synthesizing particles coated with the amphiphilic hyaluronate HA-C₁₈-1.3%, i.e. with a polymer containing 1.3 alkyl chains containing 18 carbons, per 100 glucose units, is given below.

The poly(D,L-lactic acid) (PLA, M_{w}=106 000 g/mol) and the dichloromethane (CH₂Cl₂) are Sigma-Aldrich (France) products.

10 mg of HA-C₁₈-1.3% are dissolved for 24 h with stirring in 10 ml of distilled water. 1 ml of CH₂Cl₂ containing 25 mg of PLA is added. A stable oil-in-water emulsion is prepared using a vortex for 30 s and then ultrasound at a power of 10 W in pulsed mode (50% of active cycle) for 60 s. The organic solvent is then evaporated off, with stirring, at ambient temperature and pressure, for 2 h. The aqueous suspension of particles thus obtained is washed with water by means of 3 successive centrifugations of 10 mm at 12 000 rpm.

The particles obtained under these conditions have a mean diameter of 450 nm (mean intensity diameter, determined by photon correlation spectroscopy on a Malvern 4600 device).

### Example 2

### In vitro Biological Evaluation of the Particles in Accordance with the Invention

Rat chondrocytes (cartilage cells) obtained after digestion of cartilage fragments with pronase and with collagenase are cultured in DMEM (Gibco BRL, UK) in the presence of particles obtained according to Example 1.

Two culture systems are used:

1) A conventional monolayer system applicable to all cell types and which makes it possible to determine general parameters of biocompatibility such as viability and proliferation.

The chondrocytes are dispensed in 24-well culture plates in a proportion of approximately 100 000 chrondocytes per well. A suspension of nanoparticles coated with amphiphilic HA and synthesized according to Example 1 is prepared in the DMEM culture medium so as to have, on average, approximately 10⁷ particles per ml. 1 ml of this suspension is brought into contact with the chrondocytes in the culture wells, which gives a mean ratio of approximately 100 particles per chrondocyte. The contact is maintained for 48 h.

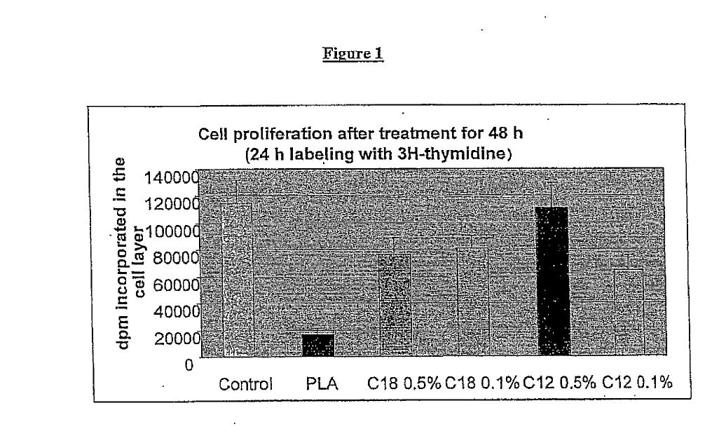

FIG. 1 shows that, after this 48 h contact with particles coated with amphiphilic HAs substituted to various degrees with chains containing 18 or 12 carbon atoms, the viability and the proliferation of the chondrocytes are similar to those obtained with the control.

On the other hand, it can be observed that, under these experimental conditions, the presence of the naked PLA particles significantly modifies these parameters.

2) A three-dimensional system: in the case of the chrondocytes, the above analysis is completed with the study of a culture in calcium alginate beads which may or may not be enriched in particles in accordance with the invention, in order to determine the proteoglycan synthesis activity of the chondrocyte in this nano- or microparticulate environment.

The cell pellets are suspended in a solution of sodium alginate at 2% in 0.9% sterile NaCl and containing the HA-coated particles, so as to have approximately 500 000 chondrocytes per ml and, on average, approximately 200 nanoparticles per chondrocyte. The resulting suspension is then deposited dropwise into a 100 mM CaCl₂ solution using a 2 ml syringe equipped with a 0.8×25 needle, which makes it possible to form beads approximately 2 mm in diameter on contact with the CaCl₂. After having been left to stand for 20 min in the CaCl₂ solution, the beads are washed twice in a row with 0.9% NaCl.

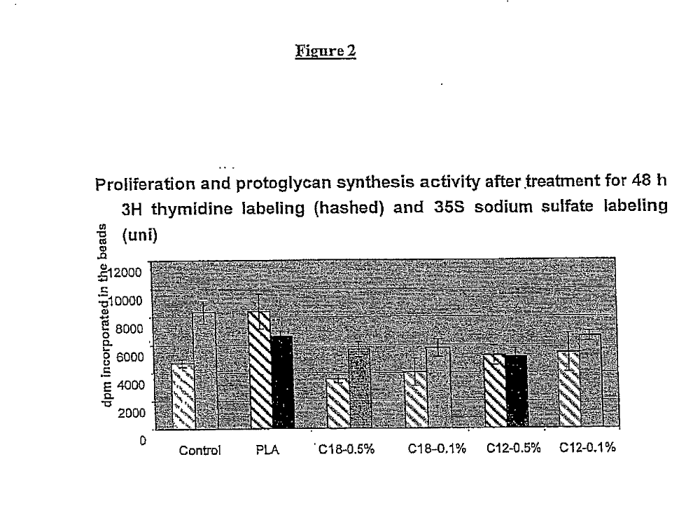

FIG. 2 shows that the contact with nanospheres coated with amphiphilic HAs substituted to various degrees with chains containing 18 or 12 carbon atoms does not significantly disturb the proliferation and the metabolic activity of the chrondocytes in this environment.

1. A method of preparing particles encapsulating a hydrophobic active substance, comprising:
preparing a single emulsion including:
dissolving a biodegradable polymer in en organic phase with the hydrophobic active substance to be encapsulated,
dissolving an amphiphilic hyaluronan in an aqueous phase, the aqueous phase being a dispersing medium for the organic phase, and
mixing the organic phase and the aqueous phase;
evaporating off an organic solvent; and
recovering the particles encapsulating the hydrophobic active substance.

2. The method of claim 1, further comprising washing the particles with water.

3. The method of claim 1, farther comprising subjecting the particles to centrifugation or lyophilization.

4. The method of claim 1, wherein the organic phase is a solvent that is barely soluble in water.

5. The method of claim 4, wherein the organic solvent is methylene chloride or ethyl acetate.

6. The method of claim 1, further comprising incorporating a diagnostic compound into the particles.

7. The method of claim 6, wherein the diagnostic compound comprises at least one substance capable of being detected by X-rays, fluorescence, ultrasound, substances nuclear magnetic resonance, or radioactivity.

8. The method of claim 6, wherein incorporating the diagnostic material occurs during formation of the particles,

9. A method of preparing particles encapsulating a hydrophilic active material, comprising:
preparing an oil-in-water type emulsion containing an organic phase and a first aqueous phase, wherein:
the organic phase contains a biodegradable organosoluble polymer, and
the first aqueous phase contains the hydrophilic active material to be encapsulated;
mixing the oil-in-water type emulsion with a second aqueous phase, wherein:
the second aqueous phase contains an amphiphilic hyaluronan, and
the mixing results in a water/oil/water double emulsion;
evaporating a solvent; and
recovering the particles encapsulating the hydrophilic active material.

10. The method of claim 9, further comprising washing the particles with water.

11. The method of claim 9, further comprising subjecting the particles to centrifugation or lyophilization.

12. The method of claim 9, further comprising incorporating a diagnostic compound into the particles.

13. The method of claim 12, wherein the diagnostic compound is at least one substance capable of being detected by X-rays, fluorescence, ultrasound, nuclear magnetic resonance, or radioactivity,

14. The method of claim 12, wherein incorporating the diagnostic material occurs during formation of the particles.

15. The method of claim 9, wherein the hydrophilic active material to be encapsulated comprises a protein or a polysaccharide,

16. A method of targeting a release of an active substance encapsulated in a particle to cells having hyaluronan-specific receptors, comprising:
surface functionalizing a particle with a hyaluronan-based coating composition comprising at least one hyaluronan or one of its derivatives;
transporting the particle to an organ to be treated; and
releasing an active substance;
wherein:
the particle includes a core containing at least one biodegradable organosoluble polymer,
the hyaluronan is a water-soluble amphiphilic hyaluronan having carboxylic groups that are partially converted to form a plurality of hydrophobic groups, and
the plurality of hydrophobic groups are anchored to and extend into the core of the particle.

17. The method of claim 16, wherein the carboxylic groups are in part esterified with at least one group selected from the group consisting of linear or branched, saturated or unsaturated, alkyl chains, and
each alkyl chain has a number of carbon atoms greater than 5 and equal to or less than 20, and
each alkyl chain has a degree of esterification of at most 50%.

18. The method of claim 16, wherein the plurality of hydrophobic groups are attached to the hyaluronan by an ester and/or amide group.

19. The method of claim 16, wherein the active substance is at least one biological active substance or at least one synthetic active substance.

20. The method of claim 19, wherein the active substance is at least one biological active substance selected from the group consisting of peptides, proteins, carbohydrates, nucleic acids, lipids, polysaccharides, antigens, enzymes, hormones, receptors, vitamins, matricial components, and mixtures thereof.
